(19) United States
(12) Patent Application Publication
(10) Pub. No.: US 2008/0147048 A1
(43) Pub. Date: Jun. 19, 2008

(54) Drain with Occlusion Removing Structure

(76) Inventor: Harvey L. Deutsch, Los Angeles, CA (US)

Correspondence Address: SHELDON MAK ROSE & ANDERSON PC, 100 Corson Street, Third Floor, PASADENA, CA, 91103-3842, US

(21) Appl. No.: 10/566,693

(22) PCT Filed: Jan. 6, 2006
(86) PCT No.: PCT/US06/00286

§ 371 Date: Jan. 31, 2006

Related U.S. Application Data
(63) Continuation-in-part of application No. PCT/US05/00508, filed on Jan. 7, 2005.
(60) Provisional application No. 60/535,224, filed on Jan. 9, 2004.
(60) Provisional application No. 60/563,597, filed on Apr. 19, 2004.

(51) Int. Cl. A61M 27/00

(52) U.S. Cl. 604/541

(57) ABSTRACT

A drainage device for draining a space or cavity defined by a wall. A method for draining a cavity or space with a drain, and unoccluding the drain. A kit for removing an occlusion from an occluded drain.

### CROSS-REFERENCE TO RELATED APPLICATIONS

This application claims priority from International Patent Application No. PCT/US05/00508 titled “Drain with Occlusion Removing Structure,” filed Jan. 7, 2005, the contents of which are incorporated in this disclosure by reference in their entirety.

### BACKGROUND

There are a variety of medical drains, such as Penrose and Jackson-Pratt drains, used post-operatively to control blood and serosanguinous discharge from operative sites and wounds. Drains are typically used following major open abdominal operations, as well as during operations involving the back, breast, chest, head, hip and vertebral column. Drains help prevent the accumulation of hematomas and seromas in post-operative sites that lead to infections, abscesses, poor wound healing and wound dehiscence.

Medical drains generally have a distal end that is placed in the surgical site and a proximal end that is brought through the skin through a stab incision adjacent to the closed surgical incision. The proximal end of the drain is usually secured to the skin by one or more sutures.

Further, the proximal end of the drain is typically connected to a suction device, such as a compressible egg-shaped, container or bulb (a “hand grenade”). Connecting the suction device to the drain applies negative pressure through the drain and into the surgical site, encouraging the egress of fluid from the surgical site through the drain. The suction device is emptied of drainage fluid, and the amount of drainage is measured periodically, such as per nursing shift or per day. The drain is removed when the amount of drainage diminishes below a set amount during a specific time.

Disadvantageously, however, medical drains are prone to occlusion with inspissated bloody or serosanguinous drainage, allowing fluids to collect at the surgical site. When a drain occludes, it must be replaced, either by an open surgical procedure or by a minimally invasive surgical procedure, such as, for example, a procedure involving interventional radiology. Drain replacement procedures add significant extra expense and increase the risk of patient morbidity during the patient's post-operative course.

Therefore, there is a need for a new device or method that addresses the issue of occluded drains that involves less expense or less risk to the patient.

### SUMMARY

According to one embodiment of the present invention, there is provided a drainage device for draining a space or cavity defined by a wall. The drainage device comprises: a) a first component comprising a proximal segment, an intermediate segment and a distal segment, where the intermediate segment and the distal segment function as an occlusion removing structure; and b) a second component that functions as a drain; where the drainage device comprises a proximal end and a distal end; where the proximal end of the proximal segment comprises an instrument for creating an opening in the wall of the space or cavity; where the intermediate segment comprises a proximal end and a distal end, and further comprises a hollow tube comprising a wall with an outer surface and an inner surface defining a central lumen; where the distal segment comprises an elongated tubular mesh comprising a proximal end, a distal end, an outer surface and an inner surface defining a central lumen; where the second component comprises a proximal end and a distal end, and further comprises a hollow tube comprising a wall with an outer surface and an inner surface defining a central lumen; where the first component and the second component are non-integrally connected; where the first component further comprises a proximal segment comprising a proximal end and a distal end; where the distal end of the proximal segment is connected to the proximal end of the intermediate segment; and where the distal end of the intermediate segment is connected to the proximal end of the distal segment. In one embodiment, the instrument is a trocar. In another embodiment, the instrument is bent or curved along its longitudinal axis. In one embodiment, the distal end of the proximal segment comprises a first connector for joining the proximal segment to the intermediate segment. In another embodiment, the inner surface of the wall of the hollow tube of the intermediate segment fits snugly over the distal end of the first connector of the proximal segment. In one embodiment, the first connector integrally joins the proximal segment to the intermediate segment.

According to another embodiment of the present invention, there is provided a drainage device for draining a space or cavity defined by a wall. The drainage device comprises: a) a first component comprising an intermediate segment and a distal segment; and b) a second component that functions as a drain; where the drainage device comprises a proximal end and a distal end; where the intermediate segment comprises a proximal end and a distal end, and further comprises a hollow tube comprising a wall with an outer surface and an inner surface defining a central lumen; where the distal segment comprises an elongated tubular mesh comprising a proximal end, a distal end, an outer surface and an inner surface defining a central lumen; where the second component comprises a proximal end and a distal end, and further comprises a hollow tube comprising a wall with an outer surface and an inner surface defining a central lumen; where the first component and the second component are non-integrally connected; and where the distal end of the intermediate segment is connected to the proximal end of the distal segment. In one embodiment, the method further comprises creating a second opening using an instrument separate from the drainage device. In another embodiment, the method further comprises closing the second opening after withdrawing the second component.

According to another embodiment of the present invention, there is provided a drainage device for draining a space or cavity defined by a wall. The drainage device comprises means for drainage and means for removing an occlusion.

In one embodiment, the drainage device further comprises a proximal end, a distal end, and a drainage device axial length between the proximal end of the drainage device and the distal end of the drainage device; and where the first component extends substantially from the proximal end of the drainage device to the distal end of the drainage device.

In one embodiment, the distal end of the intermediate segment comprises a second connector for joining the intermediate segment to the second component non-integrally.

In one embodiment, the proximal end of the distal segment is integrally joined to the distal end of the intermediate segment.

In one embodiment, the hollow tube of the second component is flexible.

In one embodiment, the hollow tube of the intermediate segment is flexible.

In one embodiment, the hollow tube of the second component further comprises a plurality of apertures extending completely through the wall of the hollow tube of the second component, from the outer surface of the hollow tube of the second component to the inner surface of the hollow tube of the second component. In a preferred embodiment, the plurality of apertures are arranged in a plurality of rows.

In one embodiment, the second component further comprises a third connector at the proximal end of the second component; and where the third connector is configured to mate non-integrally with the second connector on the distal end of the intermediate segment.

In one embodiment, the distal segment of the first component has a distal segment axial length extending from the proximal end of the tubular mesh to the distal end of the tubular mesh; where the second component has a second component axial length extending from the proximal end of the second component to the distal end of the second component; and where the distal segment axial length is between 60% and 100% of the second component axial length. In another embodiment, the distal segment of the first component has a distal segment axial length extending from the proximal end of the tubular mesh to the distal end of the tubular mesh; where the second component has a second component axial length extending from the proximal end of the second component to the distal end of the second component; and where the distal segment axial length is between 70% and 100% of the second component axial length. In another embodiment, the distal segment of the first component has a distal segment axial length extending from the proximal end of the tubular mesh to the distal end of the tubular mesh; where the second component has a second component axial length extending from the proximal end of the second component to the distal end of the second component; and where the distal segment axial length is between 80% and 100% of the second component axial length. In another embodiment, the distal segment of the first component has a distal segment axial length extending from the proximal end of the tubular mesh to the distal end of the tubular mesh; where the second component has a second component axial length extending from the proximal end of the second component to the distal end of the second component; and where the distal segment axial length is between 90% and 100% of the second component axial length. In another embodiment, the distal segment of the first component has a distal segment axial length extending from the proximal end of the tubular mesh to the distal end of the tubular mesh; where the second component has a second component axial length extending from the proximal end of the second component to the distal end of the second component; and where the distal segment axial length is between 95% and 100% of the second component axial length. In another embodiment, the distal segment of the first component has a distal segment axial length extending from the proximal end of the tubular mesh to the distal end of the tubular mesh; where the second component has a second component axial length extending from the proximal end of the second component to the distal end of the second component; and where the distal segment axial length is between 99% and 100% of the second component axial length.

In one embodiment, the drainage device further comprises a third component comprising a proximal end, a distal end, and a hollow tube comprising a wall with an outer surface and an inner surface defining a central lumen; and where the proximal end of the proximal segment fits snugly into the central lumen of the third component; and where the third component is non-integral with the first component. In another embodiment, the hollow tube of the third component is flexible.

In one embodiment, the drainage device further comprises one or more than one structure for securing the drainage device connected to the proximal end of the second component. In another embodiment, the one or more than one structure is a loop.

According to another embodiment of the present invention, there is provided a kit for removing an occlusion from an occluded drain comprising a drainage device according to the present invention, and further comprising an introducer. In one embodiment, the introducer comprises a replacement occlusion removing structure. In one embodiment, the occlusion removing structure comprises a tubular mesh. In another embodiment, the tubular mesh is preloaded into the introducer.

According to another embodiment of the present invention, there is provided a method for draining a cavity or space with a drain, and unoccluding the drain. The method comprises: a) selecting a space or cavity to be drained, where the space or cavity comprises a wall substantially defining the space or cavity to be drained; b) providing a drainage device according to the present invention; c) placing the distal end of the drainage device within the space or cavity to be drained through a first opening; d) using the proximal end of the proximal segment to create a second opening in the wall of the space or cavity to be drained, and advancing the proximal segment completely through the second opening created in the wall, before or after placing the distal end of the drainage device within the space or cavity to be drained through a first opening; e) allowing the distal end of the second component of the drainage device to remain in place for an extended period of time in order to drain drainage material from the space or cavity into the central lumen of the tubular mesh; f) allowing the second component to become occluded with drainage material; and g) withdrawing the tubular mesh proximally from the second component, thereby binding the occluding drainage material within the tubular mesh, and thereby unoccluding the second component.

According to another embodiment of the present invention, there is provided a method for draining a cavity or space with a drain, and unoccluding the drain. The method comprises: a) selecting a space or cavity to be drained, where the space or cavity comprises a wall substantially defining the space or cavity to be drained; b) providing a drainage device according to the present invention; c) placing the distal end of the drainage device within the space or cavity to be drained through a first opening; d) allowing the distal end of the second component of the drainage device to remain in place for an extended period of time in order to drain drainage material from the space or cavity into the central lumen of the tubular mesh; e) allowing the second component to become occluded with drainage material; f) withdrawing the tubular mesh proximally from the second component, thereby binding the occluding drainage material within the tubular mesh, and thereby unoccluding the second component.

In one embodiment, the space or cavity is within a human. In another embodiment, the space or cavity is created by a surgical procedure.

In one embodiment, the first opening is a naturally existing opening. In another embodiment, the first opening is a man-made opening. In another embodiment, the first opening is a surgical incision.

In one embodiment, withdrawing the tubular mesh proximally comprises rotating the intermediate segment relative to the second component, and then by axially sliding the intermediate segment proximally relative to the second component.

In one embodiment, the method further comprises separating the proximal segment of the first component from the intermediate segment after creating the second opening in the wall of the space or cavity.

In one embodiment, the drainage device further comprises a third component comprising a proximal end, a distal end, and a hollow tube comprising a wall with an outer surface and an inner surface defining a central lumen; and where the proximal end of the proximal segment fits snugly into the central lumen of the third component; where the third component is non-integral with the first component; and where the method further comprises removing the third component by axially sliding the third component proximally relative to the first component.

In one embodiment, the first opening through which the distal end of the drainage device is placed is closed after placing the distal end of the drainage device within the space or cavity. In another embodiment, the first opening is closed by suturing or stapling.

In one embodiment, the method further comprises withdrawing the second component from the space or cavity after withdrawing the tubular mesh proximally from the second component.

In one embodiment, the drainage device further comprises one or more than one structure for securing the drainage device connected to the proximal end of the second component; and the method further comprises attaching the one or more than one structure to a surface to anchor the drainage device. In another embodiment, the method further comprises detaching the one or more than one structure from the surface before withdrawing the second component. In one embodiment, the method further comprises closing the second opening after withdrawing the second component.

In one embodiment, the method further comprises attaching the proximal end of the intermediate segment to a suction device after placing the distal end of the drainage device in the space or cavity. In another embodiment, the method further comprises attaching the proximal end of the second component to a suction device after withdrawing the tubular mesh.

In one embodiment, the method further comprises cutting the distal end of the drainage device before placing the drainage device within the space or cavity. In another embodiment, cutting the distal end of the drainage device causes the cut distal end of the tubular mesh to retract slightly into the cut distal end of the second component, thereby preventing sharp points of the distal end of the tubular mesh from damaging the wall of the space or cavity during use of the drainage device.

In one embodiment, the method further comprises providing a replacement occlusion removing structure, and inserting the replacement occlusion removing structure into the second component after unoccluding the second component. In another embodiment, the method further comprises repeating the step of providing a replacement occlusion removing structure, and inserting the replacement occlusion removing structure into the second component after unoccluding the second component. In another embodiment, providing a replacement occlusion removing structure, and inserting the replacement occlusion removing structure into the second component after unoccluding the second component comprises: a) providing an introducer comprising a proximal end and a distal end; b) loading the replacement occlusion removing structure into an introducer to create a loaded introducer; c) inserting the distal end of the introducer containing the replacement occlusion removing structure into the proximal end of the second component.

### FIGURES

These and other features, aspects and advantages of the present invention will become better understood with regard to the following description, appended claims, and accompanying figures where:

FIG. 1 is a top perspective view of one embodiment of a drainage device according to the present invention comprising a drain with an occlusion removing structure;

FIG. 2 is a partial, close-up, lateral perspective view of the drainage device shown in FIG. 1;

FIG. 3 is a close-up lateral perspective view of the second component of the drainage device shown in FIG. 1 and FIG. 2;

FIG. 4 is a lateral perspective view of an alternate embodiment of the drainage device according to the present invention;

FIG. 5 is a top perspective view of the optional third component of the drainage device as shown in FIG. 1 and FIG. 2;

FIG. 6 is a lateral perspective view of an introducer according to the present invention;

FIG. 7 is a bottom perspective view of the introducer shown in FIG. 6;

FIG. 8, is a lateral perspective view of an introducer preloaded with a replacement occlusion removing structure according to the present invention; and

FIG. 9 through FIG. 15 show partial cutaway, lateral perspective views of various steps in a method of removing an occlusion from an occluded drain according to the present invention.

### DESCRIPTION

According to one embodiment of the present invention, there is provided a drainage device comprising an occlusion removing structure. According to another embodiment of the present invention, there is provided a method for draining a cavity or space with a drain, and unoccluding the drain. In a preferred embodiment, the method comprises providing a device according to the present invention. The device and method prolong the useful life of a drain, such as a medical drain, and obviate the need for replacing the drain when it becomes occluded, thereby decreasing the cost associated with drain replacement, and the risk of patient morbidity associated with drain replacement. Though the drainage device of the present invention is presented primarily in the context of a medical drain in this disclosure, the drainage device can also be used for other non-surgical purposes, as will be understood by those with skill in the art with reference to this disclosure. The device and method will now be presented in detail.

As used in this disclosure, the term “comprise” and variations of the term, such as “comprising” and “comprises,” are not intended to exclude other additives, components, integers or steps.

As used in this disclosure, two elements of a device are “integral” if they are joined together in a manner that does not allow separation of the two elements from one another by the user of the device without cutting through or destroying the element.

As used in this disclosure, two elements of a device are “non-integral” if they are joined together in a manner to allow separation of the two elements from one another by the user of the device without cutting through or destroying the element.

As used in this disclosure, the term “occlude” and variations of the term, such as “occluded,” “occluding,” and “occlusion” means a mass or clog of occluding material within the central lumen of a drain, which either partially or completely decreases the function of a drain. As will be understood by those with skill in the art with reference to this disclosure, debris being aspirated from inside a blood vessel left after removal of an embolus or plaque from the blood vessel wall by a therapy catheter does not decrease the function of an aspiration catheter aspirating the debris and, therefore, the debris within the aspiration catheter is not an occlusion within the aspiration catheter.

As used in this disclosure, the term “occlusion removing structure” refers to the intermediate segment and the distal segment of the first component, as these elements are disclosed in this disclosure.

As used in this disclosure, the term “drain” refers to the distal end of the second component.

All dimensions specified in this disclosure are by way of example only and are not intended to be limiting. Further, the proportions shown in these Figures are not necessarily to scale. As will be understood by those with skill in the art with reference to this disclosure, the actual dimensions of any device or part of a device disclosed in this disclosure will be determined by its intended use.

The devices of the present invention and their component parts comprise any suitable material for the intended purpose of the device, as will be understood by those with skill in the art with reference to this disclosure. For example, when used as a medical drain, the device will usually comprise one or more than one biocompatible material capable of being sterilized.

As will be understood by those with skill in the art with reference to this disclosure, the device of the present invention can be used for a variety of both surgical and non-surgical uses. Examples of surgical uses include biliary tubes and stents, chest tubes, decompression catheters, feeding tubes, gastrointestinal decompression catheters, gastrostomy tubes, jejunostomy tubes, mediastinal tubes, nasogastric tubes, nephrostomy catheters, percutaneous drainage catheters, peritoneal dialysis catheters such as abscess drainage catheters, vascular catheters such as venous hemodialysis catheters, and ventriculostomy tubes. Further, the device of the present invention can be used as biliary and urinary drainage catheters, such as Foley-type bladder catheters placed after bloody surgeries, such as prostate resections, where drain tubes tend to occlude with blood clots. Additionally, the device of the present invention can be utilized as a sump-type tube, that is, a drain possessing an additional lumen used to draw external air into the patient while suction is applied to the drain to prevent adhesion of the drain's side apertures to the surrounding tissues within the wound or body cavity, thereby preventing fluid drainage through the drain.

In one embodiment, the present invention comprises a drainage device. The drainage device comprises means for drainage and means for removing an occlusion. In each of the embodiments of the drainage device disclosed, the means for drainage and the means for removing an occlusion will be identified or will be understood by those with skill in the art with reference to this disclosure. In general, the means for removing an occlusion is the occlusion removing structure referred to in this disclosure (the second component), and the means for drainage is the drain (the third component).

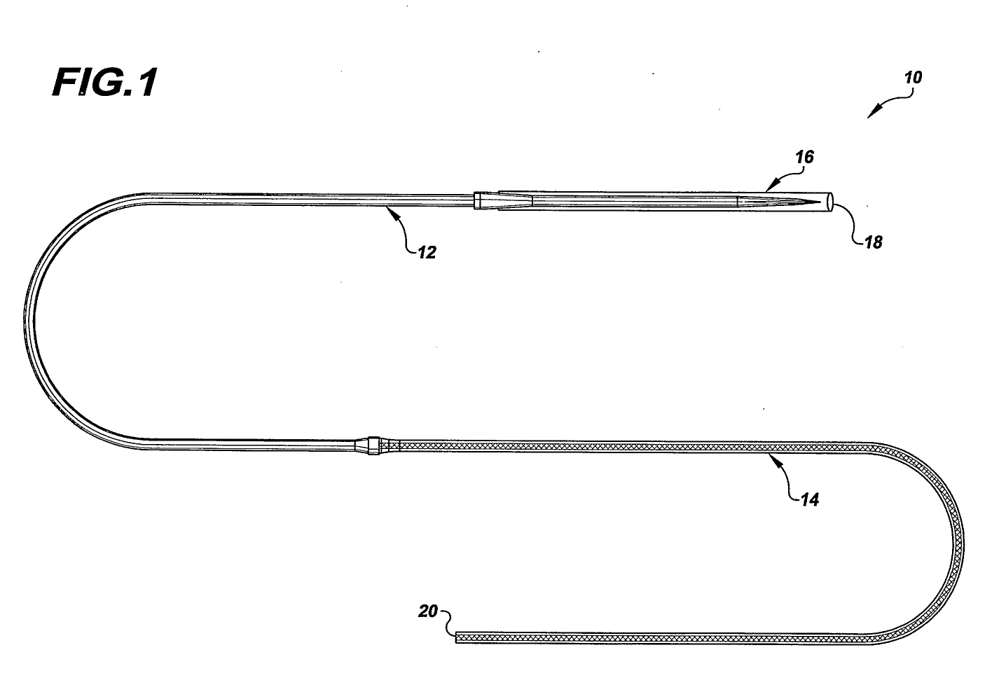

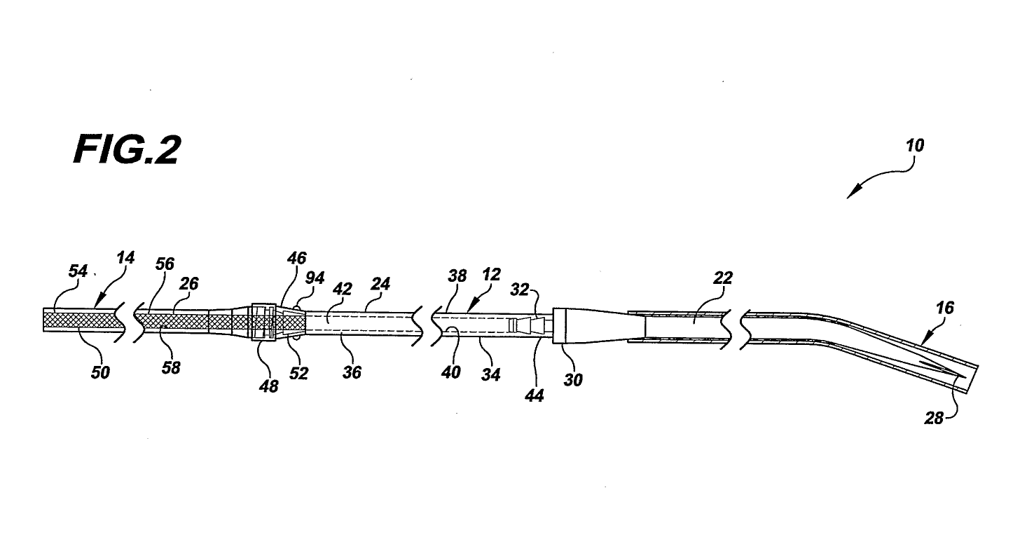

Referring now to FIG. 1 and FIG. 2, there are shown, respectively, a top perspective view of one embodiment of a drainage device according to the present invention comprising a drain with an occlusion removing structure (FIG. 1); and a partial, close-up, lateral perspective view of the drainage device shown in FIG. 1 (FIG. 2). As can be seen, in one embodiment of the present invention, the drainage device 10 comprises two components, a first component 12 and a second component 14. In a preferred embodiment, the drainage device 10 further comprises a third component 16 as seen in FIG. 1 and FIG. 2. The first component 12, the second component 14, and the third component 16 when present, are all non-integral with respect to each other as they are configured to join together in a manner to allow separation of each of the three components from one another by the user of the device without cutting through and without destroying the component. For example, in one embodiment, the first component 12 can be separated from the second component 14 by rotating the first component 12 relative to the second component 14 around the coaxial long axes of the first component 12 and the second component 14, and then by axially sliding the first component 12 proximally relative to the second component 14. The third component 16 can be separated from the first component 12 by axially sliding the third component 16 proximally relative to the first component 12. The three components will now be disclosed in greater detail.

The drainage device 10 comprises a proximal end 18 and a distal end 20, and comprises a drainage device axial length between the proximal end 28 and the distal end 20. In a preferred embodiment, the first component 12 extends substantially from the proximal end 18 of the drainage device 10 to the distal end 20 of the drainage device 10.

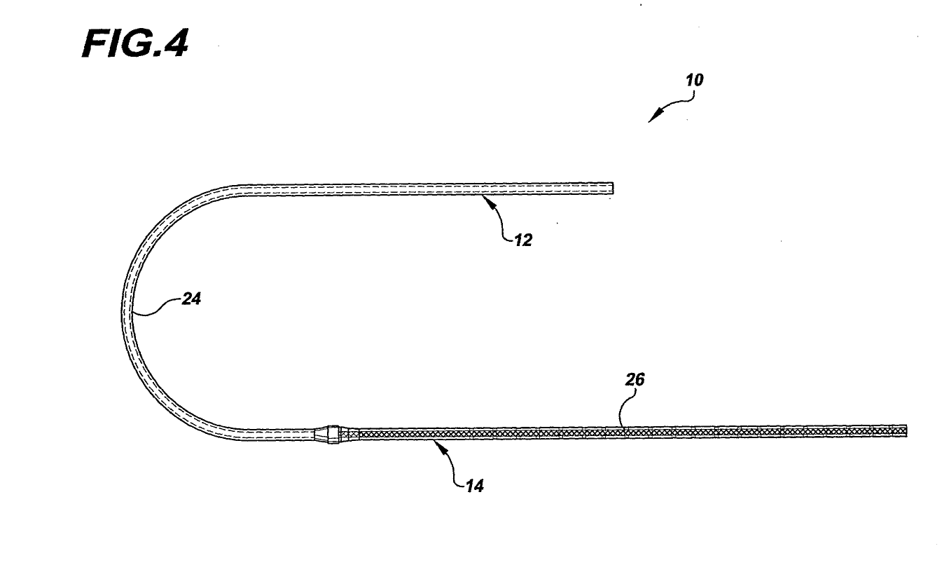

In one embodiment, the first component 12 comprises three integral segments, which are in the proximal to distal direction, a proximal segment 22, an intermediate segment 24 and a distal segment 26. In another embodiment, as will be understood by those with skill in the art with reference to this disclosure, (and as shown in FIG. 4), the first component 12 comprises the intermediate segment 24 and the distal segment 26, but does not comprise the proximal segment 22. In this latter embodiment the intermediate segment 24 and the distal segment 26, can also be called a first portion 24 (intermediate segment 24) and a second portion 26 (distal segment 26) for the sake of clarity, since this embodiment does not include a proximal segment 22.

The proximal segment 22 comprises a proximal end 28 and a distal end 30. The proximal end 28 of the proximal segment 22 comprises an instrument for creating an opening in a wall that defines a space or cavity to be drained, such as, for example, the chest wall or abdominal wall of a human, and advancing the proximal segment 22 completely through the opening created in the wall. The proximal end 28 of the proximal segment 22 can be any suitable instrument, as will be understood by those with skill in the art with reference to this disclosure, such as, for example, a rod comprising a tapered proximal end. In a preferred embodiment, the proximal segment 22 comprises a trocar, such as a surgical trocar comprising a double, triple or quatro cut bullet point. In a preferred embodiment, the proximal end 28 of the proximal segment 22 is bent or curved as shown along its longitudinal axis.

The distal end 30 of the proximal segment 22 comprises a first connector 32 for joining the proximal segment 22 to the intermediate segment 24. In a preferred embodiment, the first connector 32 integrally joins the proximal segment 22 to the intermediate segment 24. The first connector 32 can be any suitable connector, as will be understood by those with skill in the art with reference to this disclosure. In a preferred embodiment, as shown particularly in FIG. 2 and FIG. 14, the first connector 32 is a Christmas-tree type connector. In another preferred embodiment, the proximal segment 22 comprises stainless steel or another biocompatible surgical grade metal.

Referring again to FIG. 1 and FIG. 2, and particularly to FIG. 2, the intermediate segment 24 comprises a hollow tube 34 comprising a wall 36 with an outer surface 38 and an inner surface 40 defining a central lumen 42, and comprises a proximal end 44 and a distal end 46. In a preferred embodiment, the hollow tube 34 is flexible. Preferably, the proximal end 44 of the intermediate segment 24 is integrally joined to the first connector 32 of the proximal segment 22, such as, for example, by an adhesive. In one embodiment, as shown particularly in FIG. 2, the inner surface 40 of the wall 36 of the hollow tube 34 at the proximal end 44 of the intermediate segment 24 fits snugly over the distal end of the first connector 32 of the proximal segment 22.

The distal end 46 of the intermediate segment 24 comprises a second connector 48 for joining the intermediate segment 24 to the second component 14. In a preferred embodiment, the second connector 48 joins the intermediate segment 24 to the second component 14 non-integrally. The second connector 48 can be any suitable connector, as will be understood by those with skill in the art with reference to this disclosure. In a preferred embodiment, as shown particularly in FIG. 2, the second connector 48 comprises a female end of a luer-type connector; however, the second connector 48 can also be the male end of a luer-type connector, or can be any other suitable connector. In another preferred embodiment, the intermediate segment 24 comprises a biocompatible flexible polymer, such as, for example, polyethylene, or comprises silicone rubber.

Referring again to FIG. 1 and FIG. 2, and particularly to FIG. 2, the distal segment 26 comprises an elongated tubular mesh 50 comprising a proximal end 52, a distal end 54, an outer surface 56 and an inner surface 58. The proximal end 52 of the distal segment 26 is integrally joined to the second connector 48 of the intermediate segment 24 either mechanically or by chemical means, such as, for example, by an adhesive, such as, for example, an epoxy. In a preferred embodiment, the tubular mesh 50 comprises a material that can be easily cut using a pair of surgical scissors or similar instrument to a desired length as appropriate for the intended use, such as, for example, a cobalt-chromium-nickel-molybdenum-iron alloy specified by ASTM F1058 and ISO 5832-7, stainless steel or a ferrous alloy containing cobalt, chromium, nickel, molybdenum, manganese, carbon, and beryllium, such as Elgiloy®.

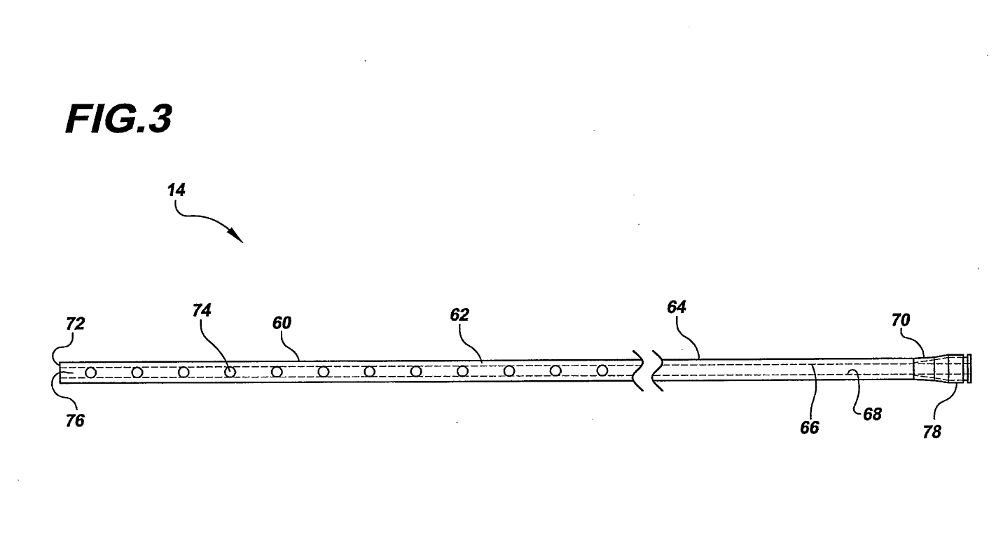

Referring now to FIG. 3, there is shown a close-up lateral perspective view of the second component of the drainage device shown in FIG. 1 and FIG. 2. As can be seen, in FIG. 1, FIG. 2 and FIG. 3, the second component 14 comprises a hollow tube 60 comprising a wall 62 with an outer surface 64, an inner surface 66 defining a central lumen 68, and comprises a proximal end 70 and a distal end 72. In a preferred embodiment, the hollow tube 60 is flexible.

The hollow tube 60 further comprises a plurality of apertures 74 extending completely through the wall 62 from the outer surface 64 to the inner surface 66, thereby creating a communication between the central lumen 68 and the space outward from the outer surface 64. The configuration, number, shape, size and spacing of the apertures 74 will vary with the intended use. In one embodiment, the hollow tube 60 comprises a plurality of rows of apertures 74. In another embodiment, the hollow tube 60 comprises one row of apertures 74. In another embodiment, the hollow tube 60 comprises two rows of apertures 74. In another embodiment, the hollow tube 60 comprises three rows of apertures 74. In another embodiment, the hollow tube 60 comprises four or more rows of apertures 74. In one embodiment, the hollow tube 60 comprises between about 4 and about 200 apertures 74. In another embodiment, the hollow tube 60 comprises between about 10 and about 100 apertures 74. In another embodiment, the hollow tube 60 comprises between about 20 and about 50 apertures 74. In another embodiment, the hollow tube 60 comprises about 30 apertures 74. In one embodiment, each aperture 74 is round. In another embodiment, each aperture 74 is oval. In another embodiment, each aperture 74 is rectangular. In another embodiment, each aperture 74 is a shape other than round, oval or rectangular. In another embodiment, at least two apertures 74 are different shapes. In one embodiment, each aperture 74 has a maximum length of between about 1 mm and 20 mm.

In another embodiment, each aperture 74 has a maximum length of between about 2 mm and 10 mm. In another embodiment, each aperture 74 has a maximum length of between about 3 mm and 5 mm. In another embodiment, at least two apertures 74 have different maximum lengths. In one embodiment, each aperture 74 is spaced apart from the center of the next closest aperture 74 of between about 0.5 mm and 50 mm. In another embodiment, each aperture 74 is spaced apart from the center of the next closest aperture 74 of between about 1 mm and 20 mm. In another embodiment, each aperture 74 is spaced apart from the center of the next closest aperture 74 of between about 2 mm and 10 mm. In another embodiment, each aperture 74 is spaced apart from the center of the next closest aperture 74 of between about 3 mm and 5 mm. In another embodiment, the spacing between the center of two apertures 74 is different from the spacing between the center of two other apertures 74. As shown in FIG. 3, by way of example only, in one embodiment, the hollow tube 60 comprises two opposing rows of round apertures 74 each about 3 mm in diameter and spaced about 1 cm apart from the center of the next closest aperture 74 in the same row. In a preferred embodiment, the hollow tube 60 also has an opening 76 at the distal end 72 of the second component 14.

The second component 14 further comprises a third connector 78 at the proximal end 70, configured to mate non-integrally with the second connector 48 on the distal end 46 of the intermediate segment 24. The third connector 78 can be any suitable connector, as will be understood by those with skill in the art with reference to this disclosure. In a preferred embodiment, as shown particularly in FIG. 3, the third connector 78 is the male end of a luer-type connector when the second connector 48 is a female end of luer-type connector; however, the third connector 78 can also be the female end of a luer-type connector when the second connector 48 is a male end of luer-type connector, or can be any other suitable connector, such as, for example, a Christmas-tree type connector or a hemostasis Y-connector. In another preferred embodiment, the second component 14 comprises a biocompatible flexible polymer, such as, for example, polyethylene, polytetrafluoroethylene and polyamide, or silicone rubber that can be easily cut using a pair of surgical scissors or similar instrument to a desired length as appropriate for the intended use. In another preferred embodiment, both the second component 14 and the tubular mesh 50 comprise material that can be easily cut using a pair of surgical scissors or similar instrument using approximately the same force, to a desired length as appropriate for the intended use.

Referring now to FIG. 4, there is shown a lateral perspective view of an alternate embodiment of the drainage device according to the present invention. In this embodiment, the first component 12 comprises the intermediate segment 24 and the distal segment 26, but does not comprise the proximal segment 22. In this embodiment, the intermediate segment 24 and the distal segment 26 can also be called a first portion 24 (intermediate segment 24) and a second portion 26 (distal segment 26) for the sake of clarity, since this embodiment does not include a proximal segment 22.

The first component 12 has a first component axial length extending from the proximal end 28 of the proximal segment (when the proximal segment 22 is present) or from the proximal end of the intermediate segment 24 (when the proximal segment is not present) to the distal end 54 of the distal segment 26. In one embodiment, the first component axial length is between 60% and 100% of the device axial length. In another embodiment, the first component axial length is between 70% and 100% of the device. In another embodiment, the first component axial length is between 80% and 100% of the device axial length. In another embodiment, the first component axial length is between 90% and 100% of the device axial length. In another embodiment, the first component axial length is between 95% and 100% of the device axial length. In another embodiment, the first component axial length is between 99% and 100% of the device axial length.

The distal segment 26 of the first component 12 has a distal segment axial length extending from the proximal end 52 of the distal segment 26 to the distal end 54 of the distal segment 26. The second component 14 has a second component axial length extending from the proximal end 70 of the second component 14 to the distal end 72 of the second component 14. In one embodiment, the distal segment axial length is between 60% and 100% of the second component axial length. In another embodiment, the distal segment axial length is between 70% and 100% of the second component axial length. In another embodiment, the distal segment axial length is between 80% and 100% of the second component axial length. In another embodiment, the distal segment axial length is between 90% and 100% of the second component axial length. In another embodiment, the distal segment axial length is between 95% and 100% of the second component axial length. In another embodiment, the distal segment axial length is between 99% and 100% of the second component axial length. For example, in one embodiment, the distal segment axial length and the second component axial length are both 50 cm.

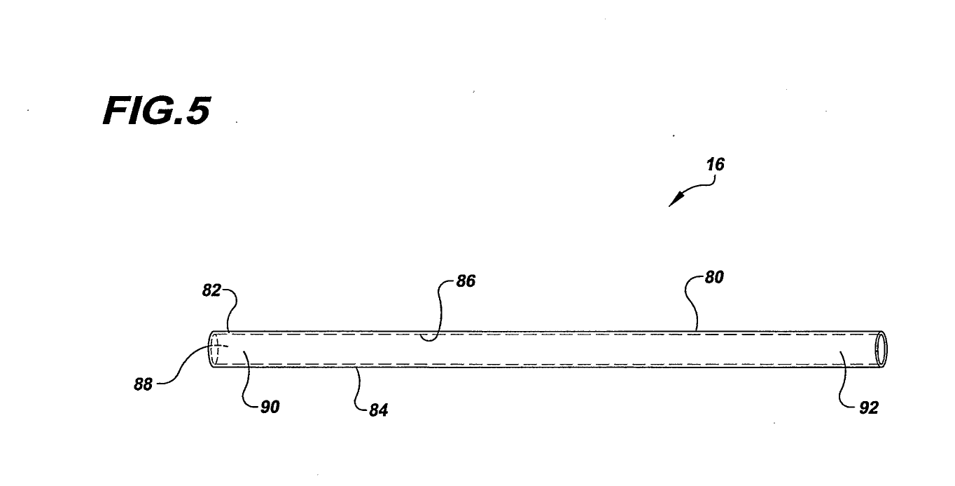

Referring now to FIG. 5, there is shown a top perspective view of the optional third component of the drainage device as shown in FIG. 1 and FIG. 2. As can be seen in FIG. 1, FIG. 2 and FIG. 5, the drainage device 10 can optionally comprise the third component 16. The third component 16 comprises a hollow tube 80 comprising a wall 82 with an outer surface 84 and an inner surface 86 defining a central lumen 88, and comprises a proximal end 90 and a distal end 92. In a preferred embodiment, the hollow tube 80 is flexible. The inner surface 86 is configured so that the proximal end 28 of the proximal segment 22 fits snugly into the central lumen 88 of the third component 16, thereby protecting the user from injury by the proximal end 28 of the proximal segment 22 during manipulation of the drain before using the proximal end 28 of the proximal segment 22 to create an opening in a wall defining a space or cavity to be drained. The wall 82 is also configured to abut the proximal end of the first connector 32 so as not to extend over the first connector 32, because the circumference of the inner surface 86 is smaller than the maximum circumference of the first connector 32. In one embodiment, the axial length of the third component 16 is approximately equal to the axial length of the proximal end 28 of the proximal segment 22. In a preferred embodiment, the axial length of the third component 16 exceeds the axial length of the proximal end 28 of the proximal segment 22 as shown in FIG. 1 and FIG. 2. In another preferred embodiment, the axial length of the third component 16 is at least 10% longer than the axial length of the proximal end 28 of the proximal segment 22. In another preferred embodiment, the axial length of the third component 16 is at least 20% longer than the axial length of the proximal end 28 of the proximal segment 22. In another preferred embodiment, the axial length of the third component 16 is at least 30% longer than the axial length of the proximal end 28 of the proximal segment 22. In another preferred embodiment, the axial length of the third component 16 is between 10% and 100% longer than the axial length of the proximal end 28 of the proximal segment 22. For example, if the proximal end 28 of the proximal segment 22 has an axial length of 16 cm, then a third component 16 having an axial length of 24 cm would be 50% longer and the axial length of the proximal end 28 of the proximal segment 22′. The section of the third component 16 extending beyond the proximal end 28 of the proximal segment 22 can be used to grasp the third component 16 and remove it from the remainder of the drainage device 10. In a preferred embodiment, the third component 16 comprises a material selected from the group consisting of polyethylene, polycarbonate, polytetrafluoroethylene, polyamide and silicone rubber. In a preferred embodiment, the third component 16 is flexible.

In another embodiment, the drainage device 10 comprises one or more than one structure 94 for securing the drainage device 10, such as one or more than one loop connected to the intermediate segment 24 or the second component. The structure 94 is used to secure the proximal end 18 of the drainage device 10 to a surface, such as the skin of a patient when the drainage device 10 is used as a medical drain, or to another surface, when the drainage device 10 is used as a non-medical drain.

As will be understood by those with skill in the art with reference to this disclosure, the drainage device 10 and its components can be any size suitable for the intended use. By way of example only, in one embodiment, both the device 10 (without the third component 16) comprises a drainage device axial length and the first component axial length of about 100 cm. The proximal segment 22 of the first component 12 has an axial length of about 16 cm, of which, the proximal end 28 of the proximal segment 22 has an axial length of about 13 cm and the distal end 30 of the proximal segment 22 has an axial length of about 3 cm. The intermediate segment 24 has an axial length of about 45 cm of which about 1 cm of the proximal end 44 of the intermediate segment 24 extends over the first connection 22. The distal segment 26 has an axial length of about 48 cm, of which about 3 cm extend into the intermediate segment 24. The second component 14 has an axial length of about 48 cm. The third component 16 has an axial length of about 15 cm with about 2 cm extending proximally beyond the proximal end 28 of the drainage device 10. In one embodiment, the inner surface 58 of the distal segment 26 has a diameter of between 1 and 20 mm. In another embodiment, the outer surface 56 of the distal segment 26 has a diameter of between about 2 mm and about 22 mm. In another embodiment, the outer surface 64 of the distal segment 26 has a diameter of between about 5 mm and about 20 mm. In another embodiment, the central lumen 68 of the second component 14 has a diameter of between about 2 mm and about 25 mm. In another embodiment, the central lumen 68 of the second component 14 has a diameter of between about 4 mm and about 22 mm. In another embodiment, the central lumen 68 of the second component 14 has a diameter of between about 6 mm and about 15 mm. In another embodiment, the central lumen 68 of the second component 14 has a diameter of between about 10 mm and about 15 mm.

The drainage device 10 of the present invention can be constructed according to standard techniques, as will be understood by those with skill in the art with reference to this disclosure.

In another embodiment, the present invention is a kit for removing an occlusion from an occluded drain. In this embodiment, the present invention comprises a drainage device 10 according to the present invention, and further comprises an introducer.

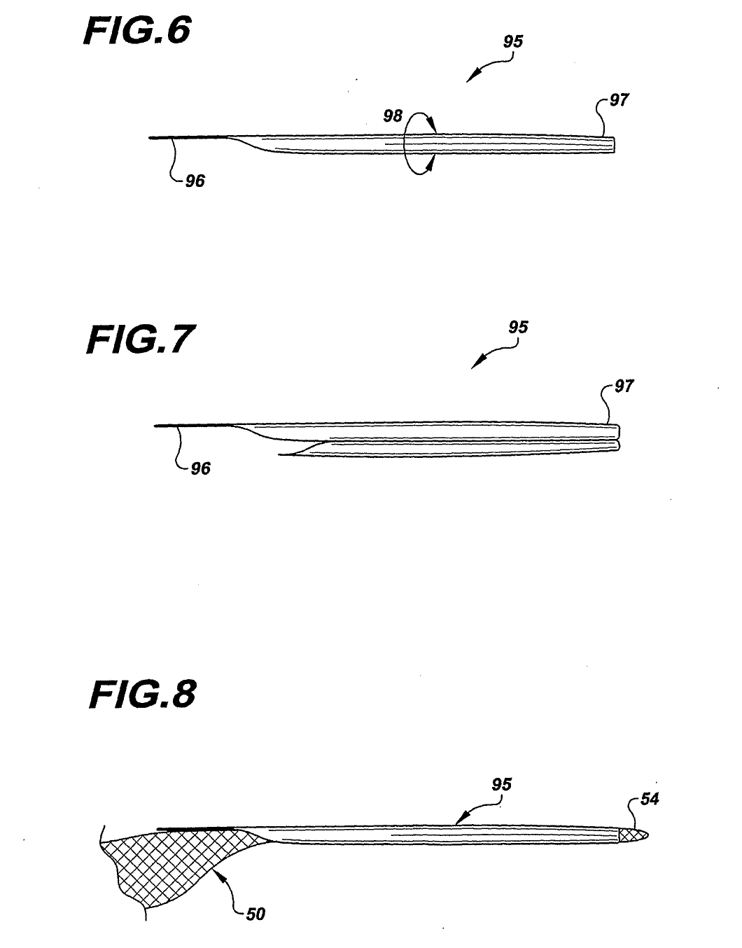

Referring now to FIGS. 6 and 7, there are shown a lateral perspective view of an introducer (FIG. 6); and a bottom perspective view of the introducer shown in FIG. 6 (FIG. 7). As can be seen, the introducer 95 comprises an elongate, tubular structure comprising a proximal end 96 and a distal end 97. The proximal end 96 is open. The distal end 97 can be open as shown, or can be closed. In a preferred embodiment, the distal end 97 is blunted. The introducer 95 further comprises a maximum external circumference 98. In one embodiment, the introducer 95 is scored down its long axis on at least one side so that the introducer 95 can be peeled away from its contents. In a preferred embodiment, the introducer 95 is flexible.

In another embodiment, the kit for removing an occlusion from an occluded drain further comprises a replacement first component 12 according to the present invention where the replacement first component 12 provided does not comprise the proximal segment 22, but consists of the intermediate segment 24 and distal segment 26 only.

Referring now to FIG. 8, there is shown a lateral perspective view of an introducer preloaded with a replacement occlusion removing structure. In a preferred embodiment, the replacement occlusion removing structure is a replacement first component 12 consisting of the intermediate segment 24 and distal segment 26. In another preferred embodiment, the replacement occlusion removing structure is a replacement tubular mesh 50 only, as shown in FIG. 8. In another preferred embodiment, the kit comprises both a drainage device 10 according to the present invention, and an introducer 95 according to the present invention. In another preferred embodiment, the kit comprises a drainage device 10 according to the present invention, and an introducer 95 preloaded with a replacement occlusion removing structure as shown in FIG. 8.

In another embodiment, the present invention comprises a method of removing an occlusion from an occluded drain.

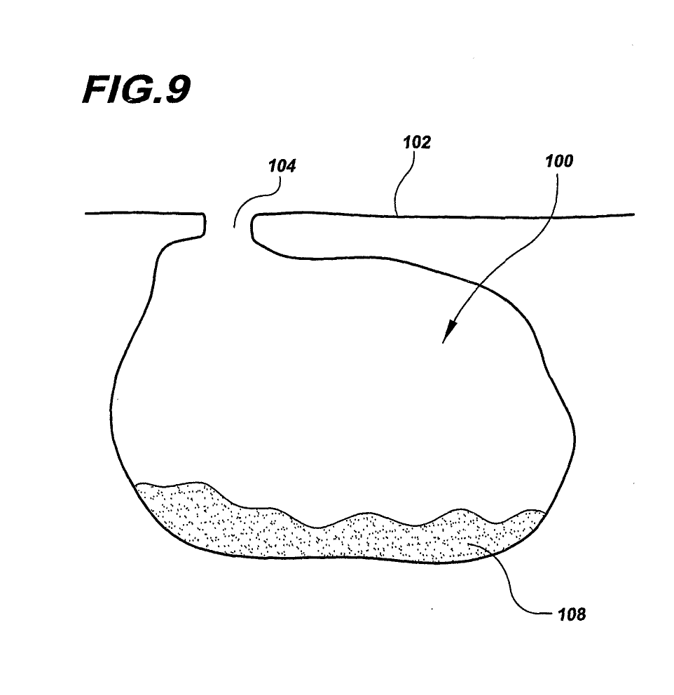

Referring now to FIG. 9 through FIG. 15, there are shown partial cutaway, lateral perspective views of various steps in a method of removing an occlusion from an occluded drain according to the present invention. As can be seen, the method comprises, first, selecting a space or cavity 100 to be drained. The space or cavity 100 comprises a wall 102 substantially defining the space or cavity 100 to be drained. In one embodiment, the space or cavity 100 is within a human. In one embodiment, the space or cavity 100 is within a non-human animal. In another embodiment, the space or cavity 100 is created by a surgical procedure. In another embodiment, the space or cavity 100 is selected from the group consisting of the abdominal cavity, the bladder, the intestines, the intracranial cavity, the mediastinum, the nasal passages, the stomach, the renal pelvis and the ureter. In another embodiment, the space or cavity 100 is created during a surgical procedure on the back, breast, chest, head, hip or vertebral column.

Next, a drainage device is provided. In one embodiment, the drainage device provided is a drainage device 10 according to the present invention, where the drainage device 10 comprises a first component 12 comprising a proximal segment 22 as well as an intermediate segment 24 and a distal segment 26. Reference is now made to FIG. 1 through FIG. 8, in addition to FIG. 9 through FIG. 15. The intermediate segment 24 and distal segment 26 are an occlusion removing structure. The second component 14 and, particularly, the distal end 72 of the second component 14 is a drain.

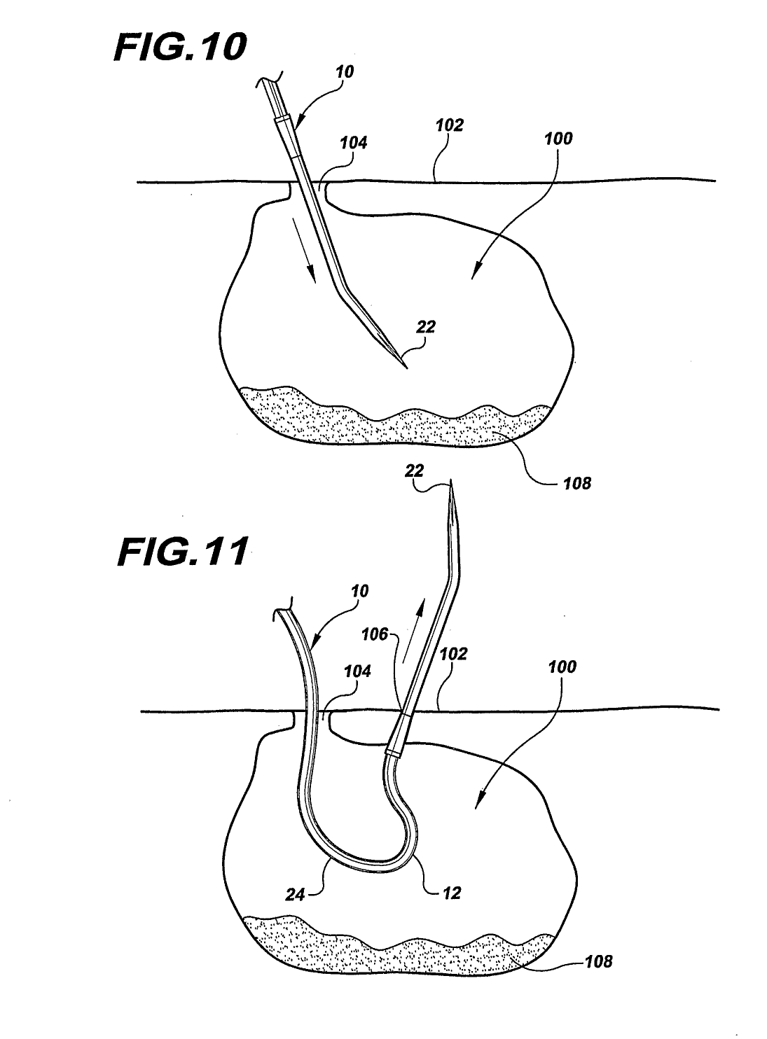

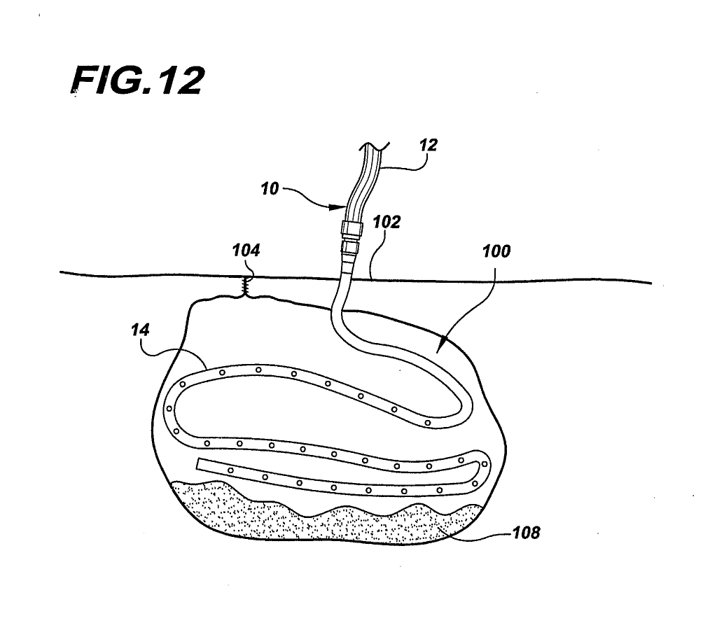

Then, the distal end 20 of the drainage device 10 is placed within the space or cavity 100 to be drained through a first opening 104. In one embodiment, placing the distal end 20 of the drainage device 10 within the space or cavity 100 comprises introducing the distal end 20 of the drainage device 10 into the space or cavity through a naturally existing opening. In another embodiment, as shown in FIG. 10, FIG. 11 and FIG. 12, the distal end 20 of the drainage device 10 is placed within the space or cavity 100 through a man-made opening, such as a surgical incision as indicated in the Figures.

Next, as shown in FIG. 11, the proximal end 28 of the proximal segment 22 is used to create a second opening 106 in the wall 102 of the space or cavity 100 to be drained, such as, for example, by creating a stab incision from inside the space or cavity 100 through the wall 102 to outside of the space or cavity 100, and the proximal segment 22 is advanced completely through the second opening 106 created in the wall 102.

Before or after placing the distal end 20 of the drainage device 10 within the space or cavity 100 to be drained, the third component 16, if present, is removed from the first component 12 by axially sliding the third component 16 proximally relative to the first component 12.

Then, the proximal segment 22 of the first component 12 is separated from the intermediate segment 24, such as, for example, by cutting the hollow tube 34 proximal to the second connector 48. This leaves at least part of the second component 14 and at least part of the occlusion removing structure consisting of the intermediate segment 24 and distal segment 26 within the space or cavity 100 as shown in FIG. 12.

In one embodiment, as shown in FIG. 12, the opening through which the distal end 20 of the drainage device 10 is placed is then closed, such as, for example, by suturing or stapling the first opening 104. In another embodiment, not shown, the drainage device 10 comprises one or more than one structure 94 for securing the drainage device 10, such as one or more than one loop connected to the first component 12. In this embodiment, the method further comprises attaching the one or more than one structure 94 to a surface to anchor the drainage device 10. In a preferred embodiment, the surface is the skin of a patient.

The distal end 72 of the second component 14 of the drainage device 10 is then allowed to remain in place for an extended period of time in order to drain drainage material 108 from the space or cavity 100, such as, for example, blood, bile, cerebrospinal fluid, gastrointestinal contents, lymphatic fluid, pus, serosanguinous fluid and urine, into the central lumen 68 of the second component 14 (and therefore, into the tubular mesh 50). In one embodiment, the distal end 72 of the second component 14 of the drainage device 10 is allowed to remain within the space or cavity 100 for between 1 hour and 30 days. In another embodiment, the distal end 72 of the second component 14 of the drainage device 10 is allowed to remain within the space or cavity 100 for between 2 hours and 20 days. In another embodiment, the distal end 72 of the second component 14 of the drainage device 10 is allowed to remain within the space or cavity 100 for between 3 hours and 10 days. In another embodiment, the distal end 72 of the second component 14 of the drainage device 10 is allowed to remain within the space or cavity 100 for between 12 hours and 10 days. In another embodiment, the distal end 72 of the second component 14 of the drainage device 10 is allowed to remain within the space or cavity 100 for between 12 hours and 5 days. In another embodiment, the distal end 72 of the second component 14 of the drainage device 10 is allowed to remain within the space or cavity 100 for between 1 day and 7 days.

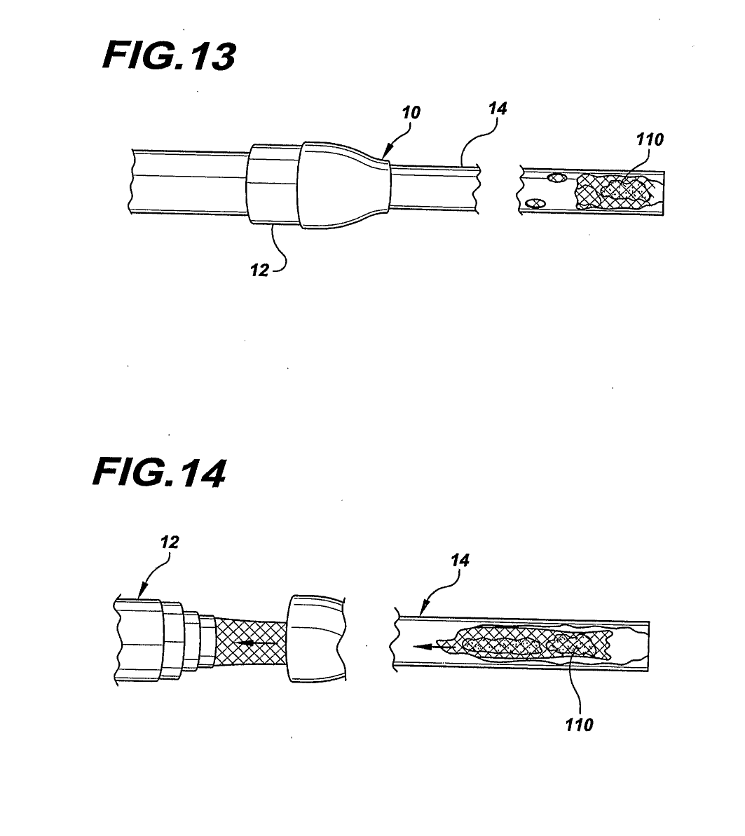

As can be seen in FIG. 13 and FIG. 14, after the distal end 72 of the second component 14 of the drainage device 10 becomes occluded with drainage material 108, the occluding drainage material 110 is removed by first, separating the intermediate segment 24 of the first component 12 from the second component 14, such as, for example, by rotating the intermediate segment 24 relative to the second component 14 around the coaxial long axes of the intermediate segment 24 and the second component 14, and then by axially sliding the intermediate segment 24 proximally relative to the second component 14. Axially sliding the intermediate segment 24 proximally relative to the second component 14 places proximal traction action on the tubular mesh 50 causing it to elongate axially and contract perpendicularly to its axis as shown in FIG. 14. This contraction tends to bind the occluding drainage material 110 within the tubular mesh 50 and keeps the occluding drainage material 108 within the tubular mesh 50 as the tubular mesh 50 is withdrawn proximally from the second component 14. Once the tubular mesh 50 is withdrawn with the occluding drainage material 110, the second component 14 is unoccluded, that is, the occluding drainage material 110 is removed from the drain 14.

In one embodiment, once the space or cavity 100 no longer needs to be drained, the drainage device 10 or what remains of the drainage device 10, such as the second component 14, in the space or cavity 100 can be withdrawn from the space or cavity 100. If one or more than one structure 94 for securing the drainage device 10 has been used to secure the drainage device 10 to a surface, the method comprises detaching the one or more than one structure 94 from the surface before withdrawing the drainage device 10 or what remains for the drainage device 10 from the space or cavity 100, such as, for example, by cutting the sutures anchoring the one or more than one structure 94 from the skin of a patient.

In one embodiment, withdrawing the drainage device 10 or what remains of the drainage device 10, such as the second component 14, from the space or cavity 100 comprises placing proximal traction on the proximal end of the drainage device 10 or what remains of the drainage device 10, such as the second component 14, thereby proximally axially sliding the drainage device 10 or what remains of the drainage device 10, such as the second component 14, through the second opening 106.

In another embodiment, the method comprises closing the second opening 106 after withdrawing the drainage device 10 or what remains of the drainage device 10, such as the second component 14, such as, for example, by suturing the second opening 106 using standard surgical techniques.

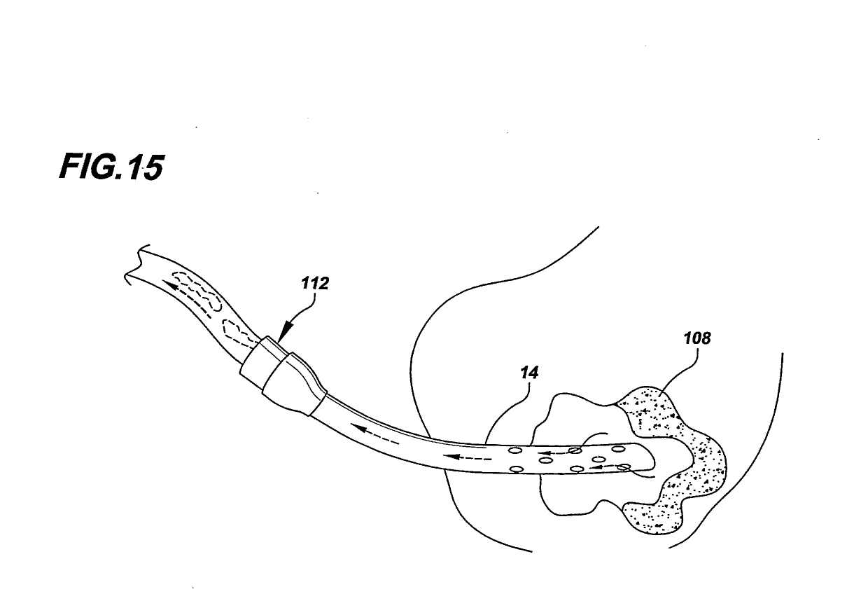

In another embodiment, the method further comprises attaching the proximal end 44 of the intermediate segment 24 to a suction device 112 after placing the distal end 20 of the drainage device 10 in the space or cavity 100. In another embodiment, as shown in FIG. 15, the method comprises attaching the proximal end 70 of the second component 14 to the suction device 112 after withdrawing the tubular mesh 50.

In another embodiment, the method further comprises cutting the distal end 20 of the drainage device 10 to shorten the axial length of the drainage device 10 before placing the drainage device 10 within the space or cavity 100. As will be understood by those with skill in the art with reference to this disclosure, cutting the distal end 20 of the drainage device 10 to shorten the axial length cuts through both the distal end 72 of the second component 14 as well as the tubular mesh 50, and leaves at least part of the distal end 72 of the second component 14 and tubular mesh 50 intact with the tubular mesh 50 still inside the central lumen 68 of the second component 14; therefore, this cutting action does not separate the first component 12 from the second component 14 in the context of this disclosure. In a preferred embodiment, the tubular mesh 50 comprises a material that can be easily cut using a pair of surgical scissors or similar instrument to a desired length as appropriate for the intended use, such as, for example, a cobalt-chromium-nickel-molybdenum-iron alloy specified by ASTM F1058 and ISO 5832-7, stainless steel or a ferrous alloy containing cobalt, chromium, nickel, molybdenum, manganese, carbon, and beryllium, such as Elgiloy® that causes the cut distal end of the tubular mesh 50 to retract slightly into the cut distal end 72 of the second component 14, thereby preventing sharp points of the distal end 54 of the tubular mesh from damaging the wall 102 of the space or cavity 100 during use of the drainage device 10.

According to another embodiment of the present invention, there is provided another method of removing an occlusion from an occluded drain. In this embodiment, the first component 12 of the drainage device 10 provided does not comprise the proximal segment 22, but consists of the intermediate segment 24 and distal segment 26 only, as shown in FIG. 4. In this embodiment, the method does not comprise using the proximal end 28 of the proximal segment 22 to create a second opening 106 in the wall 102 of the space or cavity 100 to be drained. Instead, the method comprises either creating a second opening 106 using an instrument separate from the drainage device 10, or maintaining the proximal end 18 of the drainage device 10 outside the space or cavity 100 while placing the distal end 20 of the drainage device 10 within the space or cavity 100 through a preexisting first opening 104.

According to another embodiment of the present invention, the method comprises, additionally, providing a replacement occlusion removing structure where the replacement occlusion removing structure consists of the intermediate segment 24 and distal segment 26 of a replacement first component 12. In this embodiment, the method comprises inserting the replacement occlusion removing structure into the second component 14 after removing an occlusion from the second component 14. In one embodiment, the replacement occlusion removing structure can be inserted using an introducer 95 according to the present invention, as shown in FIG. 6, FIG. 7 and FIG. 8. In one embodiment, the method comprises loading the replacement occlusion removing structure into the introducer 95 to create a loaded introducer 95 as shown in FIG. 8. In another embodiment, the method comprises providing an introducer 95 preloaded with the replacement occlusion removing structure. The method further comprises inserting the distal end 97 of the introducer 95 containing the replacement first component 12 or replacement tubular mesh 50 into the proximal end 70 of the second component 14, and advancing the introducer 95 until the distal end 97 of the introducer 95 is near the distal end 72 of the second component 14, and then, removing the introducer 95 leaving the replacement first component 12 or replacement tubular mesh 50 removed. In one embodiment, removing the introducer 95 comprises splitting the halves of the introducer 95 along its scored lines and peeling the split halves away from the replacement occlusion removing structure. The method further comprises allowing the second component 14 with the replacement occlusion removing structure to remain in place in order to drain drainage material 108 from the space or cavity 100. The remaining steps of the method are as disclosed above. In one embodiment, a second replacement occlusion removing structure is provided and inserted into the second component 14 after the first replacement occlusion removing structure is removed from the second component 14 to remove an occlusion from the second component 14. As will be understood by those with skill in the art with reference to this disclosure, additional replacement occlusion removing structures can be inserted into the second component 14 sequentially after removal of other occlusions, thereby maintaining patentcy of the drain.

Although the present invention has been discussed in considerable detail with reference to certain preferred embodiments, other embodiments are possible. Therefore, the scope of the appended claims should not be limited to the description of preferred embodiments contained in this disclosure.

What is claimed is:

1. A drainage device for draining a space or cavity defined by a wall, the drainage device comprising:
a) a first component comprising a proximal segment, an intermediate segment and a distal segment, where the intermediate segment and the distal segment function as an occlusion removing structure; and
b) a second component that functions as a drain;
where the drainage device comprises a proximal end and a distal end;
where the proximal end of the proximal segment comprises an instrument for creating an opening in the wall of the space or cavity;
where the intermediate segment comprises a proximal end and a distal end, and further comprises a hollow tube comprising a wall with an outer surface and an inner surface defining a central lumen;
where the distal segment comprises an elongated tubular mesh comprising a proximal end, a distal end, an outer surface and an inner surface defining a central lumen;
where the second component comprises a proximal end and a distal end, and further comprises a hollow tube comprising a wall with an outer surface and an inner surface defining a central lumen;
where the first component and the second component are non-integrally connected;
where the first component further comprises a proximal segment comprising a proximal end and a distal end;
where the distal end of the proximal segment is connected to the proximal end of the intermediate segment; and
where the distal end of the intermediate segment is connected to the proximal end of the distal segment.

2. The drainage device according to claim 1, where the instrument is a trocar.

3. The drainage device according to claim 1, where the instrument is bent or curved along its longitudinal axis.

4. The drainage device according to claim 1, where the distal end of the proximal segment comprises a first connector for joining the proximal segment to the intermediate segment.

5. The drainage device according to claim 4, where the inner surface of the wall of the hollow tube of the intermediate segment fits snugly over the distal end of the first connector of the proximal segment.

6. The drainage device according to claim 4, where the first connector integrally joins the proximal segment to the intermediate segment.

7. The drainage device according to claim 1, where the drainage device further comprises a proximal end, a distal end, and a drainage device axial length between the proximal end of the drainage device and the distal end of the drainage device; and
where the first component extends substantially from the proximal end of the drainage device to the distal end of the drainage device.

8. The drainage device according to claim 1, where the distal end of the intermediate segment comprises a second connector for joining the intermediate segment to the second component non-integrally.

9. The drainage device according to claim 1, where the proximal end of the distal segment is integrally joined to the distal end of the intermediate segment.

10. The drainage device according to claim 1, where the hollow tube of the second component is flexible.

11. The drainage device according to claim 1, where the hollow tube of the intermediate segment is flexible.

12. The drainage device according to claim 1, where the hollow tube of the second component further comprises a plurality of apertures extending completely through the wall of the hollow tube of the second component, from the outer surface of the hollow tube of the second component to the inner surface of the hollow tube of the second component.

13. The drainage device according to claim 12, where the plurality of apertures are arranged in a plurality of rows.

14. The drainage device according to claim 1, where the second component further comprises a third connector at the proximal end of the second component; and
where the third connector is configured to mate non-integrally with the second connector on the distal end of the intermediate segment.

15. The drainage device according to claim 1, where the distal segment of the first component has a distal segment axial length extending from the proximal end of the tubular mesh to the distal end of the tubular mesh;
where the second component has a second component axial length extending from the proximal end of the second component to the distal end of the second component; and
where the distal segment axial length is between 60% and 100% of the second component axial length.

16. The drainage device according to claim 1, where the distal segment of the first component has a distal segment axial length extending from the proximal end of the tubular mesh to the distal end of the tubular mesh;
where the second component has a second component axial length extending from the proximal end of the second component to the distal end of the second component; and
where the distal segment axial length is between 70% and 100% of the second component axial length.

17. The drainage device according to claim 1, where the distal segment of the first component has a distal segment axial length extending from the proximal end of the tubular mesh to the distal end of the tubular mesh;
where the second component has a second component axial length extending from the proximal end of the second component to the distal end of the second component; and
where the distal segment axial length is between 80% and 100% of the second component axial length.

18. The drainage device according to claim 1, where the distal segment of the first component has a distal segment axial length extending from the proximal end of the tubular mesh to the distal end of the tubular mesh;
where the second component has a second component axial length extending from the proximal end of the second component to the distal end of the second component; and
where the distal segment axial length is between 90% and 100% of the second component axial length.

19. The drainage device according to claim 1, where the distal segment of the first component has a distal segment axial length extending from the proximal end of the tubular mesh to the distal end of the tubular mesh;
where the second component has a second component axial length extending from the proximal end of the second component to the distal end of the second component; and
where the distal segment axial length is between 95% and 100% of the second component axial length.

20. The drainage device according to claim 1, where the distal segment of the first component has a distal segment axial length extending from the proximal end of the tubular mesh to the distal end of the tubular mesh;
where the second component has a second component axial length extending from the proximal end of the second component to the distal end of the second component; and
where the distal segment axial length is between 99% and 100% of the second component axial length.

21. The drainage device according to claim 1, further comprising a third component comprising a proximal end, a distal end, and a hollow tube comprising a wall with an outer surface and an inner surface defining a central lumen; and
where the proximal end of the proximal segment fits snugly into the central lumen of the third component; and
where the third component is non-integral with the first component.

22. The drainage device according to claim 21, where the hollow tube of the third component is flexible.

23. The drainage device according to claim 1, further comprising one or more than one structure for securing the drainage device connected to the proximal end of the second component.

24. The drainage device according to claim 1, where the one or more than one structure is a loop.

25. A drainage device for draining a space or cavity defined by a wall, the drainage device comprising:
a) a first component comprising an intermediate segment and a distal segment; and
b) a second component that functions as a drain;
where the drainage device comprises a proximal end and a distal end;
where the intermediate segment comprises a proximal end and a distal end, and further comprises a hollow tube comprising a wall with an outer surface and an inner surface defining a central lumen;
where the distal segment comprises an elongated tubular mesh comprising a proximal end, a distal end, an outer surface and an inner surface defining a central lumen;
where the second component comprises a proximal end and a distal end, and further comprises a hollow tube comprising a wall with an outer surface and an inner surface defining a central lumen;
where the first component and the second component are non-integrally connected; and
where the distal end of the intermediate segment is connected to the proximal end of the distal segment.

26. The drainage device according to claim 25, where the distal end of the intermediate segment comprises a second connector for joining the intermediate segment to the second component non-integrally.

27. The drainage device according to claim 25, where the proximal end of the distal segment is integrally joined to the distal end of the intermediate segment.

28. The drainage device according to claim 25, where the hollow tube of the second component is flexible.

29. The drainage device according to claim 25, where the hollow tube of the intermediate segment is flexible.

30. The drainage device according to claim 25, where the hollow tube of the second component further comprises a plurality of apertures extending completely through the wall of the hollow tube of the second component, from the outer surface of the hollow tube of the second component to the inner surface of the hollow tube of the second component.

31. The drainage device according to claim 25, where the plurality of apertures are arranged in a plurality of rows.

32. The drainage device according to claim 25, where the second component further comprises a third connector at the proximal end of the second component; and
where the third connector is configured to mate non-integrally with the second connector on the distal end of the intermediate segment.

33. The drainage device according to claim 25, where the distal segment of the first component has a distal segment axial length extending from the proximal end of the tubular mesh to the distal end of the tubular mesh;
where the second component has a second component axial length extending from the proximal end of the second component to the distal end of the second component; and
where the distal segment axial length is between 60% and 100% of the second component axial length.

34. The drainage device according to claim 25, where the distal segment of the first component has a distal segment axial length extending from the proximal end of the tubular mesh to the distal end of the tubular mesh;
where the second component has a second component axial length extending from the proximal end of the second component to the distal end of the second component; and
where the distal segment axial length is between 70% and 100% of the second component axial length.

35. The drainage device according to claim 25, where the distal segment of the first component has a distal segment axial length extending from the proximal end of the tubular mesh to the distal end of the tubular mesh;
where the second component has a second component axial length extending from the proximal end of the second component to the distal end of the second component; and
where the distal segment axial length is between 80% and 100% of the second component axial length.

36. The drainage device according to claim 25, where the distal segment of the first component has a distal segment axial length extending from the proximal end of the tubular mesh to the distal end of the tubular mesh;
where the second component has a second component axial length extending from the proximal end of the second component to the distal end of the second component; and
where the distal segment axial length is between 90% and 100% of the second component axial length.

37. The drainage device according to claim 25, where the distal segment of the first component has a distal segment axial length extending from the proximal end of the tubular mesh to the distal end of the tubular mesh;
where the second component has a second component axial length extending from the proximal end of the second component to the distal end of the second component; and
where the distal segment axial length is between 95% and 100% of the second component axial length.

38. The drainage device according to claim 25, where the distal segment of the first component has a distal segment axial length extending from the proximal end of the tubular mesh to the distal end of the tubular mesh;
where the second component has a second component axial length extending from the proximal end of the second component to the distal end of the second component; and
where the distal segment axial length is between 99% and 100% of the second component axial length.

39. The drainage device according to claim 25, further comprising a third component comprising a proximal end, a distal end, and a hollow tube comprising a wall with an outer surface and an inner surface defining a central lumen; and
where the proximal end of the proximal segment fits snugly into the central lumen of the third component; and
where the third component is non-integral with the first component.

40. The drainage device according to claim 22, where the hollow tube of the third component is flexible.

41. The drainage device according to claim 25, further comprising one or more than one structure for securing the drainage device connected to the proximal end of the second component.

42. The drainage device according to claim 25, where the one or more than one structure is a loop.

43. A drainage device for draining a space or cavity defined by a wall, the drainage device comprising means for drainage and means for removing an occlusion.

44. A kit for removing an occlusion from an occluded drain comprising a drainage device according to claim 1, and further comprising an introducer.

45. A kit for removing an occlusion from an occluded drain comprising a drainage device according to claim 25, and further comprising an introducer.

46. The kit according to claim 44, where the introducer comprises a replacement occlusion removing structure.

47. The kit according to claim 45, where the introducer comprises a replacement occlusion removing structure.

48. A kit for removing an occlusion from an occluded drain comprising an introducer and comprising an occlusion removing structure;
where the occlusion removing structure comprises a tubular mesh.

49. The kit of claim 48, where the tubular mesh is preloaded into the introducer.

50. A method for draining a cavity or space with a drain, and unoccluding the drain, the method comprising:
a) selecting a space or cavity to be drained, where the space or cavity comprises a wall substantially defining the space or cavity to be drained;
b) providing a drainage device according to claim 1;
c) placing the distal end of the drainage device within the space or cavity to be drained through a first opening;
d) using the proximal end of the proximal segment to create a second opening in the wall of the space or cavity to be drained, and advancing the proximal segment completely through the second opening created in the wall, before or after placing the distal end of the drainage device within the space or cavity to be drained through a first opening;
e) allowing the distal end of the second component of the drainage device to remain in place for an extended period of time in order to drain drainage material from the space or cavity into the central lumen of the tubular mesh;
f) allowing the second component to become occluded with drainage material; and
g) withdrawing the tubular mesh proximally from the second component, thereby binding the occluding drainage material within the tubular mesh, and thereby unoccluding the second component.

51. The method of claim 50, where the space or cavity is within a human.

52. The method of claim 50, where the space or cavity is created by a surgical procedure.

53. The method of claim 50, where the first opening is a naturally existing opening.

54. The method of claim 50, where the first opening is a man-made opening.

55. The method of claim 50, where the first opening is a surgical incision.

56. The method of claim 50, where withdrawing the tubular mesh proximally comprises rotating the intermediate segment relative to the second component, and then by axially sliding the intermediate segment proximally relative to the second component.

57. The method of claim 50, further comprising separating the proximal segment of the first component from the intermediate segment after creating the second opening in the wall of the space or cavity.

58. The method of claim 50, where the drainage device further comprises a third component comprising a proximal end, a distal end, and a hollow tube comprising a wall with an outer surface and an inner surface defining a central lumen; and
where the proximal end of the proximal segment fits snugly into the central lumen of the third component;
where the third component is non-integral with the first component; and
where the method further comprises removing the third component by axially sliding the third component proximally relative to the first component.

59. The method of claim 50, where the first opening through which the distal end of the drainage device is placed is closed after placing the distal end of the drainage device within the space or cavity.

60. The method of claim 59, where the first opening is closed by suturing or stapling.

61. The method of claim 50, further comprising withdrawing the second component from the space or cavity after withdrawing the tubular mesh proximally from the second component.

62. The method of claim 50, where the drainage device further comprises one or more than one structure for securing the drainage device connected to the proximal end of the second component; and
where the method further comprises attaching the one or more than one structure to a surface to anchor the drainage device.

63. The method of claim 62, further comprising detaching the one or more than one structure from the surface before withdrawing the second component.

64. The method of claim 50, further comprising closing the second opening after withdrawing the second component.

65. The method of claim 50, further comprising attaching the proximal end of the intermediate segment to a suction device after placing the distal end of the drainage device in the space or cavity.

66. The method of claim 50, further comprising attaching the proximal end of the second component to a suction device after withdrawing the tubular mesh.

67. The method of claim 50, further comprising cutting the distal end of the drainage device before placing the drainage device within the space or cavity.

68. The method of claim 67, where cutting the distal end of the drainage device causes the cut distal end of the tubular mesh to retract slightly into the cut distal end of the second component, thereby preventing sharp points of the distal end of the tubular mesh from damaging the wall of the space or cavity during use of the drainage device.

69. The method of claim 50, further comprising providing a replacement occlusion removing structure, and inserting the replacement occlusion removing structure into the second component after unoccluding the second component.

70. The method of claim 69, further comprising repeating the step of providing a replacement occlusion removing structure, and inserting the replacement occlusion removing structure into the second component after unoccluding the second component.

71. The method of claim 69, where providing a replacement occlusion removing structure, and inserting the replacement occlusion removing structure into the second component after unoccluding the second component comprises:
a) providing an introducer comprising a proximal end and a distal end;
b) loading the replacement occlusion removing structure into an introducer to create a loaded introducer; and
c) inserting the distal end of the introducer containing the replacement occlusion removing structure into the proximal end of the second component.

72. A method for draining a cavity or space with a drain, and unoccluding the drain, the method comprising:
a) selecting a space or cavity to be drained, where the space or cavity comprises a wall substantially defining the space or cavity to be drained;
b) providing a drainage device according to claim 25;
c) placing the distal end of the drainage device within the space or cavity to be drained through a first opening;
d) allowing the distal end of the second component of the drainage device to remain in place for an extended period of time in order to drain drainage material from the space or cavity into the central lumen of the tubular mesh;
e) allowing the second component to become occluded with drainage material; and
f) withdrawing the tubular mesh proximally from the second component, thereby binding the occluding drainage material within the tubular mesh, and thereby unoccluding the second component.

73. The method of claim 72, where the space or cavity is within a human.

74. The method of claim 72, where the space or cavity is created by a surgical procedure.

75. The method of claim 72, where the first opening is a naturally existing opening.

76. The method of claim 72, where the first opening is a man-made opening.

77. The method of claim 72, where the first opening is a surgical incision.

78. The method of claim 72, where withdrawing the tubular mesh proximally comprises rotating the intermediate segment relative to the second component, and then by axially sliding the intermediate segment proximally relative to the second component.

79. The method of claim 72, further comprising withdrawing the second component from the space or cavity after withdrawing the tubular mesh proximally from the second component.

80. The method of claim 72, where the drainage device further comprises one or more than one structure for securing the drainage device connected to the proximal end of the second component; and
where the method further comprises attaching the one or more than one structure to a surface to anchor the drainage device.

81. The method of claim 80, further comprising detaching the one or more than one structure from the surface before withdrawing the second component.

82. The method of claim 72, further comprising attaching the proximal end of the intermediate segment to a suction device after placing the distal end of the drainage device in the space or cavity.

83. The method of claim 72, further comprising attaching the proximal end of the second component to a suction device after withdrawing the tubular mesh.

84. The method of claim 72, further comprising cutting the distal end of the drainage device before placing the drainage device within the space or cavity.

85. The method of claim 84, where cutting the distal end of the drainage device causes the cut distal end of the tubular mesh to retract slightly into the cut distal end of the second component, thereby preventing sharp points of the distal end of the tubular mesh from damaging the wall of the space or cavity during use of the drainage device.

86. The method of claim 72, further comprising providing a replacement occlusion removing structure, and inserting the replacement occlusion removing structure into the second component after unoccluding the second component.

87. The method of claim 86, further comprising repeating the step of providing a replacement occlusion removing structure, and inserting the replacement occlusion removing structure into the second component after unoccluding the second component.

88. The method of claim 72, further comprising closing the first opening after placing the distal end of the drainage device within the space or cavity.

89. The method of claim 89, where the first opening is closed by suturing or stapling.

90. The method of claim 72, further comprising creating a second opening using an instrument separate from the drainage device.

91. The method of claim 90, further comprising closing the second opening after withdrawing the second component.
